(19) United States
(12) Patent Application Publication
(10) Pub. No.: US 2022/0340650 A1
(43) Pub. Date: Oct. 27, 2022

(54) COMBINATION THERAPY WITH CGRP ANTAGONISTS

(71) Applicants: Abnijeet JAKATE, Edison, NJ (US); Antonia PERICLOU, Rutherford, NJ (US); Ramesh BOINPALLY, Princeton, NJ (US); Allergan Pharmaceuticals International Limited, Dublin (IE)

(72) Inventors: Abhijeet Jakate, Edison, NJ (US); Antonia Periclou, Rutherford, NJ (US); Ramesh Boinpally, Princeton, NJ (US)

(21) Appl. No.: 17/763,925

(22) PCT Filed: Sep. 25, 2020
(86) PCT No.: PCT/US2020/052891

§ 371 Date: Mar. 25, 2022

Related U.S. Application Data
(60) Provisional application No. 63/070,424, filed on Aug. 26, 2020.
(60) Provisional application No. 62/905,635, filed on Sep. 25, 2019.

(51) Int. Cl. C07K 16/18; C07K 16/28; A61K 31/4545; A61K 39/395; A61P 25/06

(52) U.S. Cl. CPC C07K 16/18; C07K 16/28; A61K 31/4545; A61K 39/3955; A61P 25/06; C07K 2317/24; C07K 2317/21; A61K 2039/505

(57) ABSTRACT

Disclosed are methods for treating headache, migraine and related symptoms by administering a long acting calcitonin gene related peptide (CGRP) antagonist and a short acting CGRP antagonist. Specifically, the disclosure provides a method for treating, preventing, alleviating, or reducing the frequency of occurrence of headache in a patient in need thereof, comprising administering to the patient: (a) a first calcitonin gene related peptide antagonist (CGRP antagonist) and (b) a second CGRP-antagonist, wherein the first CGRP antagonist is an antibody, and the second CGRP antagonist has a plasma half-life in humans of at most about 60 hours.

### FIELD

The application is related to medicaments and methods for treating migraine, headache and related symptoms. The present application is directed to combination therapy by a long acting calcitonin-gene-related-peptide (hereafter referred to as CGRP) antagonist with a second short acting CGRP antagonist.

### BACKGROUND

Migraine is a common neurological disorder that affects up to 16% of the adult population in Western countries. It is characterized by attacks, often of disabling headache, which can be associated with sensory, visual, or language (aura), associated symptoms such as nausea and/or vomiting, phonophobia and photophobia, as well as dizziness and cognitive symptoms. Although the triptan group of drugs provides effective relief from acute migraine attacks for many patients, a substantial number (up to 40% in the case of oral triptans) of affected individuals are unresponsive. (Edvinsson L. Br J Clin Pharmacol. 80:2 193-199, 2015) Migraine is a highly disabling neurological condition, and preventative treatment still remains problematic, due to aspecificity of the majority of the currently available prophylactic drugs. (Adele M., et. al., Journal of Pain Research 2017:10 2751-2760, 2018).

During a migraine attack the trigeminal ganglion and trigeminal nerve endings secrete many neuropeptides that are involved in migraine pathophysiology. One of these neuropeptides is Calcitonin-gene-related peptide (CGRP), which plays a crucial role in migraine pathophysiology and several agents aimed at blocking its activity are currently being developed for the treatment of migraine, including a class of compounds knows as gepants and monoclonal antibodies (mAbs) against CGRP. For example, erenumab is a new promising candidate for the treatment of episodic migraine. (Adele M., et. al.) Erenumab is a fully human monoclonal antibody that binds to the CGRP receptor. (Goadsby P. J. et. al., N Engl J Med 2017; 377:2123-2132). There is a need for new and improved methods for the treatment and prevention of migraine and associated symptoms.

### SUMMARY

The application provides methods for treating, preventing, alleviating or reducing the frequency of occurrence of migraine, headache, or a symptom of migraine in patients by the use of two or more antagonists of calcitonin gene-related peptide (CGRP-antagonists). In some embodiments, the patient is administered a CGRP-antagonist that has a long half-life followed by an immediate release CGRP-antagonist. In some embodiments, the long acting CGRP-antagonist is an injectable formulation while the immediate release CGRP-antagonist is an orally administered formulation. In some embodiments, the immediate release CGRP-antagonist is ubrogepant, atogepant, rimegepant, telcagepant, zavegepant, or a pharmaceutically acceptable salt, ester or prodrug thereof. In some embodiments, the long acting CGRP-antagonist is an antibody selected from galcanezumab, fremanezumab, eptinezumab, and erenumab.

### BRIEF DESCRIPTION OF THE DRAWINGS

FIG. 1 provides an overview of the study design for a two-part, multi-center, open-label phase 1b trial (NCT04179474) evaluating the impact of erenumab or galcanezumab on the pharmacokinetics, safety, and tolerability of ubrogepant in patients with migraine.

FIG. 2 shows mean (SD) plasma ubrogepant concentrations following administration alone and coadministration with erenumab in the NCT04179474 study.

FIG. 3 shows mean (SD) plasma ubrogepant concentrations following administration alone and coadministration with galcanezumab.

### DESCRIPTION

The application provides methods for treating, preventing, alleviating or reducing the frequency of occurrence of migraine, headache, or a symptom of migraine in patients by the use antagonists of calcitonin gene-related peptide (CGRP-antagonists), wherein the patient is concurrently undergoing treatment with a long acting CGRP-antagonist or a CGRP-antagonist with long plasma half-life.

For example, the immediate release or short acting CGRP-antagonist may have a plasma half-life in humans of at most about 60 hours, preferably 30, 24, 18, 12, 6 or 3 hours. For example, the long acting CGRP-antagonist may have a plasma half-life in humans of more than about 60 hours. In some embodiments, the short acting antibody CGRP-antagonist is selected from ubrogepant, atogepant and rimegepant, telcagepant, zavegepant, or a pharmaceutically acceptable salt, ester or prodrug thereof.

In some embodiments, the long acting CGRP-antagonist can be an injectable antibody (anti-CGRP antibody) that is administered every one week or two weeks or three weeks or four weeks or five weeks or six weeks or two months or three months or four months or five months or six months or once a year. In some embodiments, the long acting antibody CGRP-antagonist is selected from galcanezumab, fremanezumab, eptinezumab or erenumab, or a formulation comprising said antibody. In embodiments, the long-acting CGRP antagonist is an antibody that targets the CGRP receptor, such as erenumab. In embodiments, the long-acting CGRP antagonist is an antibody that targets the CGRP ligand, such as galcanezumab. In embodiments, the long-acting CGRP antagonist is not metabolized by CYP enzymes. For example, neither erenumab or galcanezumab is metabolized by CYP enzymes.

In embodiments, ubrogepant, atogepant, telcagepant, zavegepant, or rimegepant can be used as a supplement to the antibody therapy. In embodiments, ubrogepant, atogepant, telcagepant, or rimegepant can be used to reduce the frequency of the use of antibody based therapy with galcanezumab, fremanezumab, eptinezumab or erenumab. A long-acting CGRP antagonist may be used for preventive treatment of migraines, and the short-acting CGRP antagonist (e.g., ubrogepant, atogepant, telcagepant, zavegepant, or rimegepant) may be used for the acute treatment of breakthrough migraine attacks. For example, a patient with migraine may be taking a monoclonal antibody (such as galcanezumab, fremanezumab, eptinezumab or erenumab) for preventative treatment may be prescribed ubrogepant for the acute treatment of breakthrough migraine attacks.

In some embodiments, the combination therapy is administered to the patient at a dose sufficient to reduce the number of monthly migraine headache days experienced by the patient as compared to the number of monthly migraine headache days prior to treatment or the number of monthly migraine headache days experienced by a patient receiving the treatment with a single CGRP antagonist alone.

In some embodiments, the headache is a migraine headache, a cluster headache, a hemicrania continua headache, a new daily persistent headache, a chronic daily headache, a tension headache, a chronic tension headache, or a combination thereof. In some embodiments, the migraine is migraine without aura. In some embodiments, the migraine is menstrual migraine, familial hemiplegic migraine, migraine with brainstem aura, vestibular migraine, migraine triggered by a traumatic brain injury or chronic migraine.

In some embodiments, the anti-CGRP antibody or fragment thereof is administered at a dosage that is about 20% or 30% or 40% or 50% or 60% or 70% or 80% lower than the recommended dosage for the anti-CGRP antibody monotherapy. In some embodiments, the anti-CGRP antibody or antigen-binding fragment thereof is administered to a peripheral nerve, a cranial nerve, or combinations thereof. CGRP-antagonists can be administered by any method that allows the antagonist to reach the intended targets.

For example, erenumab can be administered weekly, biweekly, monthly, every two months, every three months, every four months, every five months or every six months at a dosage of about 5 mg to about 500 mg.

Erenumab can be administered parenterally, subcutaneously or by peripheral administration. (Brauser D., Phase 3 STRIVE and ARISE Trials Show Efficacy, Safety for Erenumab in Migraine Prevention, Medscape Medical News, 2017) In one embodiment, erenumab is administered to trigeminal nerves.

In some embodiments, erenumab can be administered to the patient over the course of a set treatment period or indefinitely. (U.S. Patent Publication No. 20160311913) The treatment period can begin upon administration of a first dose of erenumab and continue while they have symptoms.

The combination therapy with ubrogepant, atogepant, rimegepant, zavegepant, or telcagepant includes administration prior to, during or after the treatment period with erenumab. The treatment period can vary and in some embodiments, lasts only one day where the antibody is administered, and in some embodiments, can continue indefinitely while the patient continues to suffer from headache or migraine. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In some embodiment, erenumab is administered in a pharmaceutical composition comprising a buffer (preferably an acetate buffer), a surfactant (preferably polysorbate 20 or polysorbate 80), and a stabilizing agent (preferably sucrose). In one particular embodiment, the treatment period is at least about 6 months and produces a statistically significant reduction in the frequency, duration, or severity of migraine headache in the patient as compared to patients treated with erenumab alone.

In one embodiment, erenumab can be administered subcutaneously at a dose of about 5 mg to about 500 mg every one, two, three, four, five, six, seven, eight, nine or ten weeks.

In one embodiment, erenumab can be administered subcutaneously at a dose of about 10 mg to about 200 mg every one, two, three, four, five, six, seven, eight, nine or ten weeks.

In one embodiment, erenumab can be administered subcutaneously at a dose of about 25 mg to about 150 mg every one, two, three, four, five, six, seven, eight, nine or ten weeks.

In one embodiment, erenumab can be administered subcutaneously at a dose of about 90 mg to about 120 mg every one, two, three, four, five, six, seven, eight, nine or ten weeks.

In one embodiment, erenumab can be administered subcutaneously at a dose of about 50 mg to about 60 mg every one, two, three, four, five, six, seven, eight, nine or ten weeks.

In one embodiment, erenumab can be administered subcutaneously at a dose of about 70 mg every one, two, three, four, five, six, seven, eight, nine or ten weeks.

In one embodiment, erenumab can be administered subcutaneously at a dose of about 140 mg every one, two, three, four, five, six, seven, eight, nine or ten weeks.

In one embodiment, erenumab can be administered subcutaneously at a monthly dose of about 140 mg. In embodiments, the 140 mg dose may be administered once monthly as two consecutive injections of 70 mg each.

In one embodiment, erenumab can be administered subcutaneously at a monthly dose of about 70 mg.

In one embodiment, erenumab can be administered subcutaneously at a dose of about 140 mg every two months.

In one embodiment, erenumab can be administered subcutaneously at a dose of about 70 mg every two months.

In one embodiment, erenumab can be administered subcutaneously at a dose of about 140 mg every three months.

In one embodiment, erenumab can be administered subcutaneously at a dose of about 70 mg every three months.

In one embodiment, an anti-CGRP antibody galcanezumab can be administered weekly, biweekly, monthly, every two months, every three months, every four months, every five months or every six months at a dosage of about 5 mg to about 500 mg.

In some embodiments, galcanezumab can be administered to the patient over the course of a set treatment period. The treatment period can begin upon administration of a first dose galcanezumab and ends upon administration of a final dose of galcanezumab. The combination therapy with ubrogepant, atogepant, telcagepant, zavegepant, or rimegepant includes administration prior to, during or after the treatment period with galcanezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of migraine headache in the patient as compared to patients treated with galcanezumab alone.

In one embodiment, galcanezumab is administered subcutaneously at a dose of about 10 mg to about 500 mg every one, two, three, four, five, six, seven, eight, nine or ten weeks.

In one embodiment, galcanezumab is administered subcutaneously at a dose of about 50 mg to about 300 mg every one, two, three, four, five, six, seven, eight, nine or ten weeks.

In one embodiment, galcanezumab is administered subcutaneously at a dose of about 75 mg to about 250 mg every one, two, three, four, five, six, seven, eight, nine or ten weeks.

In one embodiment, galcanezumab is administered subcutaneously at a dose of about 75 mg to about 100 mg every one, two, three, four, five, six, seven, eight, nine or ten weeks.

In one embodiment, galcanezumab is administered subcutaneously at a dose of about 150 mg to about 220 mg every one, two, three, four, five, six, seven, eight, nine or ten weeks.

In one embodiment, galcanezumab is administered subcutaneously at a dose of about 120 mg every one, two, three, four, five, six, seven, eight, nine or ten weeks.

In one embodiment, galcanezumab is administered subcutaneously at a dose of about 240 mg every one, two, three, four, five, six, seven, eight, nine or ten weeks.

In one embodiment, galcanezumab is administered subcutaneously at a monthly dose of about 240 mg.

In one embodiment, galcanezumab is administered subcutaneously at a monthly dose of about 120 mg.

In embodiments, galcanezumab may be administered as a 240 mg loading dose, followed by monthly doses of 120 mg. In embodiments, the 240 mg loading dose may be administered as two consecutive injections of 120 mg each.

In embodiments, galcanezumab may be administered subcutaneously at a monthly dose of about 300 mg. In embodiments, the 300 mg dose may be administered as three consecutive injections of 100 mg each.

In one embodiment, galcanezumab is administered subcutaneously at a dose of about 240 mg every two months.

In one embodiment, galcanezumab is administered subcutaneously at a dose of about 120 mg every two months.

In one embodiment, galcanezumab is administered subcutaneously at a dose of about 240 mg every three months.

In one embodiment, galcanezumab is administered subcutaneously at a dose of about 120 mg every three months.

In some embodiments, fremanezumab can be administered to the patient over the course of a set treatment period. (Silberstein, S. D., et. al., N Engl J Med 2017; 377:2113-22.) The treatment period can begin upon administration of a first dose fremanezumab and ends upon administration of a final dose of fremanezumab. The combination therapy with ubrogepant, atogepant, telcagepant, zavegepant, or rimegepant includes administration prior to, during or after the treatment period with fremanezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of migraine headache in the patient as compared to patients treated with fremanezumab alone.

In one embodiment, fremanezumab is administered subcutaneously at a dose of about 100 mg to about 1000 mg every one, two, three, four, five, six, seven, eight, nine or ten weeks.

In one embodiment, fremanezumab is administered subcutaneously at a dose of about 150 mg to about 700 mg every one, two, three, four, five, six, seven, eight, nine or ten weeks.

In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 150 mg to about 200 mg every one, two, three, four, five, six, seven, eight, nine or ten weeks.

In one embodiment, fremanezumab is administered subcutaneously at a dose of about 150 mg to about 500 mg every one, two, three, four, five, six, seven, eight, nine or ten weeks.

In one embodiment, fremanezumab is administered subcutaneously at a dose of about 225 mg every one, two, three, four, five, six, seven, eight, nine or ten weeks.

In one embodiment, fremanezumab is administered subcutaneously at a dose of about 450 mg every one, two, three, four, five, six, seven, eight, nine or ten weeks.

In one embodiment, fremanezumab is administered subcutaneously at a dose of about 675 mg every one, two, three, four, five, six, seven, eight, nine or ten weeks.

In one embodiment, fremanezumab is administered subcutaneously at a monthly dose of about 225 mg.

In one embodiment, fremanezumab is administered subcutaneously at a monthly dose of about 450 mg.

In one embodiment, fremanezumab is administered subcutaneously at a monthly dose of about 675 mg.

In one embodiment, fremanezumab is administered subcutaneously at a dose of about 225 mg every two months.

In one embodiment, fremanezumab is administered subcutaneously at a dose of about 450 mg every two months.

In one embodiment, fremanezumab is administered subcutaneously at a dose of about 225 mg every three months.

In one embodiment, fremanezumab is administered subcutaneously at a dose of about 450 mg every three months.

In one embodiment, fremanezumab is administered subcutaneously at a dose of about 675 mg every three months. In embodiments, the 675 mg dose may be administered as three consecutive injections of 225 mg each.

In some embodiments, eptinezumab can be administered to the patient over the course of a set treatment period. The treatment period can begin upon administration of a first dose eptinezumab and ends upon administration of a final dose of eptinezumab. The combination therapy with ubrogepant, atogepant, telcagepant, zavegepant, or rimegepant includes administration prior to, during or after the treatment period with eptinezumab. The treatment period may comprise from about 1 month to about 36 months, such as about 2 months, about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, or about 33 months. In some embodiments, the treatment period is about 6 months. In other embodiments, the treatment period is about 7 months. In yet other embodiments, the treatment period is about 12 months. In certain embodiments, the treatment period can be longer than 36 months, such as 48 or 60 or 64 months or more. In one particular embodiment, the treatment period is at least about 6 months and produces a statistically significant reduction in the frequency, duration, or severity of migraine headache in the patient as compared to patients treated with eptinezumab alone.

In one embodiment, eptinezumab is administered subcutaneously at a dose of about 50 mg to about 1000 mg every one, two, three, four, five, six, seven, eight, nine or ten weeks.

In one embodiment, eptinezumab is administered subcutaneously at a dose of about 100 mg to about 700 mg every one, two, three, four, five, six, seven, eight, nine or ten weeks.

In one embodiment, eptinezumab is administered subcutaneously at a dose of about 200 mg to about 500 mg every one, two, three, four, five, six, seven, eight, nine or ten weeks.

In one embodiment, eptinezumab is administered subcutaneously at a dose of about 250 mg to about 350 mg every one, two, three, four, five, six, seven, eight, nine or ten weeks.

In one embodiment, eptinezumab is administered subcutaneously at a dose of about 300 mg every one, two, three, four, five, six, seven, eight, nine or ten weeks.

In one embodiment, eptinezumab is administered subcutaneously at a monthly dose of about 100 mg.

In one embodiment, eptinezumab is administered subcutaneously at a monthly dose of about 200 mg.

In one embodiment, eptinezumab is administered subcutaneously at a monthly dose of about 300 mg.

In one embodiment, eptinezumab is administered subcutaneously at a dose of about 100 mg every two months.

In one embodiment, eptinezumab is administered subcutaneously at a dose of about 200 mg every two months.

In one embodiment, eptinezumab is administered subcutaneously at a dose of about 300 mg every two months.

In one embodiment, eptinezumab is administered subcutaneously at a dose of about 100 mg every three months.

In one embodiment, eptinezumab is administered subcutaneously at a dose of about 200 mg every three months.

In one embodiment, eptinezumab is administered subcutaneously at a dose of about 300 mg every three months.

In some embodiments, ubrogepant can be administered to the patient over the course of a set treatment period or indefinitely while the patient needs treatment. The treatment can be given over the life of the patent's disease continuously or intermittently as needed. In some embodiments, the treatment period includes a single administration of ubrogepant to a patient undergoing treatment with an anti-CGRP antibody selected from galcanezumab, fremanezumab, eptinezumab and erenumab. In some embodiments, the treatment period continues where the patient is administered ubrogepant continuously or intermittently for a year or more. The treatment period can begin upon administration of a first dose of ubrogepant and continue until the patient is administered ubrogepant on a regular or intermittent basis.

In some embodiments, ubrogepant is administered at an oral dose of about 5 to about 500 mg once, twice or three times a day. For example, in embodiments, ubrogepant is administered at an oral dose of about 50 mg to about 200 mg once, twice, or three times a day. In embodiments, ubrogepant is administered at an oral dose of about 50 mg to about 100 mg once, twice, or three times a day.

In some embodiments, ubrogepant is administered at an oral dose of about 25 mg once, twice or three times a day.

In some embodiments, ubrogepant is administered at an oral dose of about 50 mg once, twice or three times a day.

In some embodiments, ubrogepant is administered at an oral dose of about 100 mg once, twice or three times a day.

In some embodiments, ubrogepant is administered at an oral dose of about 200 mg once, twice or three times a day.

In some embodiments, atogepant can be administered to the patient over the course of a set treatment period or indefinitely while the patient needs treatment. The treatment can be given over the life of the patent's disease continuously or intermittently as needed. In some embodiments, the treatment period includes a single administration of atogepant to a patient undergoing treatment with galcanezumab, fremanezumab, eptinezumab or erenumab. In some embodiments, the treatment period continues where the patient is administered atogepant continuously or intermittently for a year or more. The treatment period can begin upon administration of a first dose atogepant and continue until the patient is administered atogepant on a regular or intermittent basis.

In some embodiments, atogepant is administered at an oral dose of about 5 to about 500 mg once, twice or three times a day.

In some embodiments, atogepant is administered at an oral dose of about 25 mg once, twice or three times a day.

In some embodiments, atogepant is administered at an oral dose of about 50 mg once, twice or three times a day.

In some embodiments, atogepant is administered at an oral dose of about 100 mg once, twice or three times a day.

In some embodiments, atogepant is administered at an oral dose of about 200 mg once, twice or three times a day.

In some embodiments, rimegepant can be administered to the patient over the course of a set treatment period or indefinitely while the patient needs treatment. The treatment can be given over the life of the patent's disease continuously or intermittently as needed. In some embodiments, the treatment period includes a single administration of rimegepant to a patient undergoing treatment with galcanezumab, fremanezumab, eptinezumab or erenumab. In some embodiments, the treatment period continues where the patient is administered rimegepant continuously or intermittently for a year or more. The treatment period can begin upon administration of a first dose rimegepant and continue until the patient is administered rimegepant on a regular or intermittent basis.

In some embodiments, rimegepant is administered at an oral dose of about 5 to about 500 mg once, twice or three times a day.

In some embodiments, rimegepant is administered at an oral dose of about 25 mg once, twice or three times a day.

In some embodiments, rimegepant is administered at an oral dose of about 50 mg once, twice or three times a day.

In embodiments, rimegepant is administered at an oral dose of about 75 mg once, twice, or three times a day. In embodiments, rimegepant is administered at an oral dose of about 75 mg once daily.

In some embodiments, rimegepant is administered at an oral dose of about 100 mg once, twice or three times a day.

In some embodiments, rimegepant is administered at an oral dose of about 200 mg once, twice or three times a day.

In some embodiments, rimegepant is administered at an oral dose of about 5 to about 500 mg once, twice or three times a day.

In some embodiments, rimegepant is administered at an oral dose of about 25 mg once, twice or three times a day.

In some embodiments, rimegepant is administered at an oral dose of about 50 mg once, twice or three times a day.

In some embodiments, rimegepant is administered at an oral dose of about 100 mg once, twice or three times a day.

In some embodiments, rimegepant is administered at an oral dose of about 200 mg once, twice or three times a day.

In some embodiments, zavegepant (previously called vazegepant) can be administered to the patient over the course of a set treatment period or indefinitely while the patient needs treatment. The treatment can be given over the life of the patent's disease continuously or intermittently as needed. In some embodiments, the treatment period includes a single administration of zavegepant to a patient undergoing treatment with galcanezumab, fremanezumab, eptinezumab or erenumab. In some embodiments, the treatment period continues where the patient is administered zavegepant continuously or intermittently for a year or more. The treatment period can begin upon administration of a first dose zavegepant and continue until the patient is administered zavegepant on a regular or intermittent basis.

In embodiments, zavegepant is administered intranasally. In embodiments, zavegepant is administered subcutaneously, through inhalation, or orally.

In some embodiments, zavegepant is administered at a dose of about 5 to about 500 mg once, twice or three times a day.

In embodiments, zavegepant is administered at a dose of about 10 mg once, twice, or three times a day.

In embodiments, zavegepant is administered at a dose of about 20 mg once, twice, or three times a day.

In some embodiments, zavegepant is administered at a dose of about 25 mg once, twice or three times a day.

In some embodiments, zavegepant is administered at a dose of about 50 mg once, twice or three times a day.

In some embodiments, zavegepant is administered at a dose of about 100 mg once, twice or three times a day.

In some embodiments, zavegepant is administered at a dose of about 200 mg once, twice or three times a day.

In some embodiments, zavegepant is administered at a dose of about 5 to about 500 mg once, twice or three times a day.

In some embodiments, zavegepant is administered at a dose of about 25 mg once, twice or three times a day.

In some embodiments, zavegepant is administered at a dose of about 50 mg once, twice or three times a day.

In some embodiments, zavegepant is administered at a dose of about 100 mg once, twice or three times a day.

In some embodiments, zavegepant is administered at a dose of about 200 mg once, twice or three times a day.

In some embodiments, the combination therapy reduces the frequency, severity and/or duration of migraine headache in patients in need thereof. In some embodiments, the combination therapy reduces the frequency of one or more symptoms or side effects of migraine; for example, sinusitis, nausea or nasopharyngitis, in comparison with a patient undergoing treatment with anti-CGRP antibody treatment alone. In some embodiments, the frequency of symptoms associated with one or more stages of migraine selected from prodrome, aura, headache or postdrome is reduced. For example, one or more frequency of symptoms associated with prodrome stage of migraine selected from photophobia, appetite changes, cognition and concentration difficulties, cold extremities, diarrhea or other bowel changes, excitement or irritability, fatigue, frequent urination, memory changes, weakness, yawning and stretching is reduced. In some embodiments, one or more frequency of symptoms associated with aura stage of migraine selected from seeing bright spots or flashes of light, vision loss, seeing dark spots, tingling sensations, speech problems, aphasia, and tinnitus is reduced. In some embodiments, one or more frequency of symptoms associated with attack stage of migraine selected from photophobia, gastric stasis, pulsating or throbbing pain on one or both sides of the head, extreme sensitivity to light, sounds, or smells, worsening pain during physical activity, nausea and vomiting, abdominal pain or heartburn, loss of appetite, lightheadedness, blurred vision, and fainting is reduced.

In some embodiments, the combination therapy of ubrogepant, atogepant, telcagepant, zavegepant, or rimegepant and an anti-CGRP antibody described herein is administered to a patient undergoing treatment with one or more additional medications for the treatment of headache. For example, the patient can be undergoing treatment with one or more additional medications selected from opioid analgesics, Cox-2 inhibitors, barbiturates, sodium channel modulators, potassium channel modulators, calcium channel blockers, local anesthetics, monoamine oxidase inhibitors, leukotriene LTD4 receptor blocker, substance P antagonists, 5-HT3 antagonists and NMDA antagonists. For example, the additional medication can be selected from aspirin, ibuprofen, naproxen, acetaminophen, diclofenac, flurbiprofen, meclofenamate, isometheptene, indomethacin; codeine, morphine, hydrocodone, acetyldihydrocodeine, oxycodone, oxymorphone, papaverine, fentanyl, alfentanil, sufentanil, remifentanil, tramadol, prochlorperazine, celecoxib, rofecoxib, meloxicam, piroxicam, JTE-522, L-745,337, NS398, deracoxib, valdecoxib, lumiracoxib, etoricoxib, parecoxib, 4-(4-cyclohexyl-2-methyloxazol-5-yl)-2 fluorobenzenesulfonimide, (2-(3,5-difluorophenyl)-3-(4-(methylsulfonyl)phenyl)-2 cyclopenten-1-one, N-[2-(cyclohexyloxy)-4-nitrophenyl]methanesulfonamide, 2-(3,4 difluorophenyl)-4-(3-hydroxy-3-methylbutoxy)-5-[4-(methylsulfonyl) phenyl]-3(2H) pyridazinone, 2-[(2,4-dichloro-6-methylphenyl)amino]-5-ethyl-benzeneacetic acid, (3Z) 3-[(4-chlorophenyl) [4-(methyl sulfonyl)phenyl]methylene]dihydro-2(3H)-furanone, (S)-6,8-dichloro-2-(trifluoromethyl)-2H-1-benzopyran-3-carboxylic acid, amobarbital, butalbital, cyclobarbital, pentobarbital, allobarbital, methylphenobarbital, phenobarbital, secobarbital, vinylbital, verapamil, diltiazem, Nifedipine, lidocaine, tetracaine, prilocaine, bupivacaine, mepivacaine, etidocaine, procaine, benzocaine, phenelzine, isocarboxazid, dichloralphenazone, nimodipine, metoclopramide, capsaicin receptor agonists, captopril, tiospirone, a steroid, caffeine, metoclopramide, domperidone, scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth sub salicylate, buclizine, cinnarizine, cyclizine, diphenidol, dolasetron, domperidone, dronabinol, droperidol, haloperidol, metoclopramide, nabilone, thiethylperazine, trimethobenzamide, and eziopitant, Meclizine, domperidone, ondansetron, tropisetron granisetron dolasetron, hydrodolasetron, palonosetron, alosetron, cilansetron, cisapride, renzapride metoclopramide, galanolactone, phencyclidine, ketamine, dextromethorphan, and isomers, pharmaceutically acceptable salts, esters, conjugates, or prodrugs thereof.

In some embodiments, the immediate release CGRP-antagonist can be administered orally, sublingually, transdermally, subcutaneously, intravenously, intradermally or intramuscularly. In some embodiments, the long acting CGRP-antagonist can be administered orally, sublingually, transdermally, subcutaneously, intravenously, intradermally or intramuscularly.

In one embodiment, the anti-CGRP antibody can be administered intravenously. The intravenous formulation can contain a tonicity modifier to avoid crenation or hemolysis of red blood cells, and/or to mitigate or avoid pain and discomfort to the patient. Preferably, the formulation to be administered to the patient has an effective osmotic pressure that is approximately the same as that of the blood of the patient. Tonicity modifiers can be non-ionic tonicity modifiers such as glycerol, sorbitol, mannitol, sucrose, propylene glycol or dextrose, or a mixture thereof. Preferably the non-ionic tonicity modifier is dextrose, sucrose or mannitol, or a mixture thereof. Aqueous pharmaceutical formulations for intravenous administration generally can have a pH of from 3 to 9.

### Definitions

As used herein, the words or terms set forth below have the following definitions:

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value.

“Administration”, or “to administer” means the step of giving (i.e. administering) a pharmaceutical composition to a subject, or alternatively a subject receiving a pharmaceutical composition. The combination therapy disclosed herein can be locally administered by various methods. For example, intramuscular, intradermal, subcutaneous administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“CGRP”, abbreviated for Calcitonin-Gene-Related-Peptide, as used herein encompasses any member of the calcitonin family, including any calcitonin gene related peptide and analogs, calcitonin, amylin, adrenomedullin and their analogs.

“CGRP antagonist” refers to any molecule that exhibits any one or more of the following characteristics: (a) bind to CGRP or CGRP-R and the binding results in a reduction or inhibition of CGRP activity; (b) block CGRP from binding to its receptor(s); (c) block or decrease CGRP receptor activation; (d) inhibit CGRP biological activity or downstream pathways mediated by CGRP signaling function; (e) increase clearance of CGRP; and (f) inhibit or reduce CGRP synthesis, production or release. CGRP antagonists include but are not limited to antibodies to CGRP, antibodies to the CGRP-R, small molecules that antagonize CGRP, and small molecules that antagonize CGRP-R.

“Combination therapy” refers to a treatment wherein a short acting or immediate release CGRP antagonist and a long acting CGRP antagonist are administered either simultaneously or sequentially, by a similar administration route or by different administration routes.

“Effective amount” as applied to the biologically active ingredient means that amount of the ingredient which is generally sufficient to effect a desired change in the subject. For example, where the desired effect is a reduction in duration or intensity of a headache, migraine and related symptoms, an effective amount of the ingredient is that amount which causes at least a substantial reduction in duration or intensity of the headache, migraine and related symptoms, and without resulting in significant toxicity.

“Intramuscular” or “intramuscularly” means into or within (as in administration or injection of a CGRP antagonist into) a muscle.

“Local administration” means direct administration of a pharmaceutical at or to the vicinity of a site on or within an animal body, at which site a biological effect of the pharmaceutical is desired, such as via, for example, intramuscular or intra- or subdermal injection or topical administration. Topical administration is a type of local administration in which a pharmaceutical agent is applied to a patient's skin.

“Peripheral administration” means administration by means of a peripheral location on a mammal. Peripheral administration includes subdermal, intranasal, intramuscular, intradermal, transdermal, and subcutaneous administration.

“Pharmaceutical composition” means a composition comprising an active pharmaceutical ingredient, such as, for example, ubrogepant, for example, a stabilizer or excipient or the like. A pharmaceutical composition is therefore a formulation which is suitable for diagnostic or therapeutic administration to a subject, such as a human patient. The pharmaceutical composition can be, for example, in a lyophilized or vacuum dried condition, a solution formed after reconstitution of the lyophilized or vacuum dried pharmaceutical composition, or as a solution or solid which does not require reconstitution.

“Pharmacologically acceptable excipient” is synonymous with “pharmacological excipient” or “excipient” and refers to any excipient that has substantially no long term or permanent detrimental effect when administered to mammal and encompasses compounds such as, e.g., stabilizing agent, a bulking agent, a cryo-protectant, a lyo-protectant, an additive, a vehicle, a carrier, a diluent, or an auxiliary. An excipient generally is mixed with an active ingredient, or permitted to dilute or enclose the active ingredient and can be a solid, semi-solid, or liquid agent. Non-limiting examples of pharmacologically acceptable excipients can be found in, e.g., Pharmaceutical Dosage Forms and Drug Delivery Systems (Howard C. Ansel et al., eds., Lippincott Williams & Wilkins Publishers, 7^{th} ed. 1999); Remington: The Science and Practice of Pharmacy (Alfonso R. Gennaro ed., Lippincott, Williams & Wilkins, 20^{th} ed. 2000); Goodman & Gilman's The Pharmacological Basis of Therapeutics (Joel G. Hardman et al., eds., McGraw-Hill Professional, 10^{th} ed. 2001); and Handbook of Pharmaceutical Excipients (Raymond C. Rowe et al., APhA Publications, 4^{th} edition 2003), each of which is hereby incorporated by reference in its entirety.

“Stabilizers” can include excipients and can include protein and non-protein molecules.

“Symptoms” related to migraine refers to any and all medically recognized symptoms of migraine. Non-limiting examples of such symptoms include vertigo, nausea, vomiting, fatigue, aura, photophobia, and phonophobia. Symptoms can also include constipation, mood changes (for example from depression to euphoria), food cravings, neck stiffness, increased thirst and urination, frequent yawning, visual phenomena (such as seeing various shapes, bright spots or flashes of light) vision loss, pins and needles sensations in an arm or leg, weakness or numbness in the face or one side of the body, difficulty speaking, hearing noises or music, uncontrollable jerking or other movements, pain on one or both sides of the head, pain that throbs or pulses, sensitivity to light, sound, and sometimes smell and touch, nausea and vomiting,

“Therapeutic formulation” means a formulation can be used to treat and thereby alleviate a disorder or a disease, such as, for example, a headache or headache-associated symptoms.

“Treating” means to alleviate (or to eliminate) at least one symptom of a condition or disorder, such as, for example, a headache, or headache-associated symptoms, either temporarily or permanently.

Methods and medicaments for treating, preventing, alleviating or reducing the frequency of occurrence of migraine, headaches, and related symptoms according to the present disclosure can comprise a long acting CGRP antagonist in combination with a short acting CGRP antagonist. The CGRP antagonists can be administered in a therapeutically effective amount to alleviate at least one symptom of a neurological disorder, including headache, migraine and related symptoms.

Non-limiting examples of centrally mediated disorders include migraine, epilepsy, chronic pain (such as central sensitization chronic pain, central post stroke pain, regional pain, phantom limb pain, or demyelinating disease pain), reflex sympathetic dystrophy, allodynic states; chronic neurological conditions in which kindling is part of the disease process; mood disorders (including bipolar disease) and movement; muscle-related and neuromuscular disorders.

### EXAMPLES

A two-part, multi-center, open-label, phase 1b trial (NCT04179474) in 40 adults (aged 18-50 years) diagnosed with migraine was conducted to evaluate the impact of erenumab or galcanezumab on the pharmacokinetics (PK), safety, and tolerability of ubrogepant in patients with migraine.

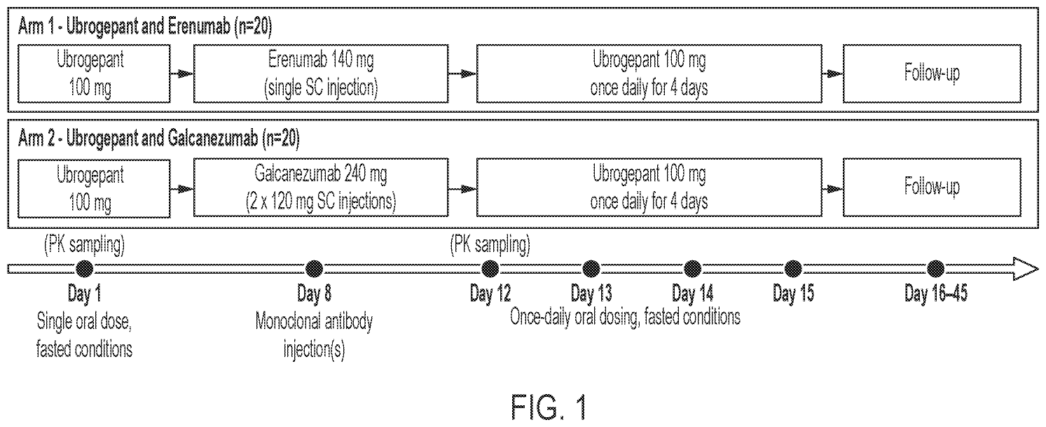

This open-label, multicenter trial enrolled adults diagnosed for greater than or equal to one year with migraine not on preventive treatment. Participants were assigned to an erenumab arm (Part 1) or a galcanezumab arm (Part 2). In each arm, participants received ubrogepant 100 mg on day 1 (without an antibody). On day 8, participants received the assigned monoclonal antibody: single subcutaneous (SC) erenumab 140 mg injection (Part 1) or galcanezumab 240 mg (2 SC 120 mg injections) (Part 2). Ubrogepant 100 mg was administered once daily on days 12-15. Participants were followed for 30 days. A summary of the study design is provided in FIG. 1.

Of 40 enrolled participants, 20 were included in part 1 (n=20 ubrogepant, n=19 erenumab, and n=19 co-administered ubrogepant and erenumab) and 20 were included in part 2 (n=20 ubrogepant, n=20 galcanezumab, and n=19 co-administered ubrogepant and galcanezumab).

PK parameters, derived from plasma ubrogepant concentrations in blood collected at prespecified time points over 24 hours post-dose on days 1 and 12, included area under the curve from time 0 to time t (AUC₀₋ₜ) or to infinity (AUC_{0-∞}); peak plasma concentration (Cₘₐₓ), time to Cₘₐₓ (Tₘₐₓ); and apparent terminal half life (t_{1/2}). Safety outcomes included incidence of adverse events (AEs; daily and through follow-up), and laboratory values, vital signs, and electrocardiogram (ECG) results (at prespecified timepoints pre- and post-dose). Log-transformed Cₘₐₓ, AUC₀₋ₜ, and AUC₀₋₂₈ , for ubrogepant with and without erenumab or galcanezumab with 90% CI were constructed.

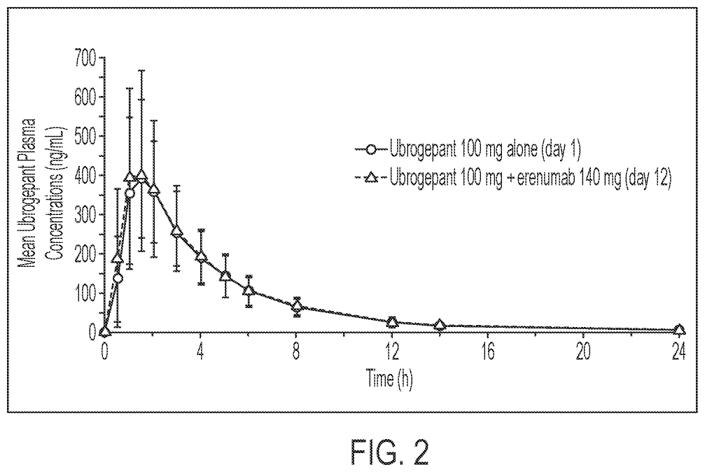

Mean ubrogepant plasma concentration-time profiles with and without coadministration of erenumab are shown in FIG. 2. Mean PK parameters are summarized below in Table 1.

TABLE 1
Ubrogepant Pharmacokinetics: Coadministration with Erenumab

| PK Parameter, | Ubrogepant Alone | | Ubrogepant + Erenumab | |
|---|---|---|---|---|
| mean (SD) | (n = 20) | | (n = 19) | |
| Cₘₐₓ, ng/mL | 459.3 | (168.6) | 486.8 | (201.5) |
| AUC₀₋ₜ, ng · h/mL | 1841.4 | (490.2) | 1960.0 | (639.5) |
| AUC_{0-∞}, ng · h/mL | 1878.3 | (497.8) | 1993.4 | (639.2) |
| tₘₐₓ, h^{a} | 1.5 | (1.0-3.0) | 1.5 | (0.5-3.0) |
| t_{1/2}, h | 5.3 | (1.0) | 4.6 | (0.9) |
| V_{Z}/F, L | 437.0 | (158.1) | 375.8 | (161.4) |
| CL/F, L/h | 57.4 | (17.2) | 54.7 | (16.3) |

* Median (range)
AUC_{0-∞}, area under the plasma concentration-time curve from time 0 to infinity; AUC₀₋ₜ, area under the concentration-time curve from time 0 to t; CL/F, apparent total body clearance of drug from plasma after extravascular administration; Cₘₐₓ, peak plasma concentration; PK, pharmacokinetic; t_{1/2}, elimination half-life; tₘₐₓ, time to peak plasma concentration; V_{Z}/F, apparent volume of distribution during the terminal phase after extravascular administration.

A comparison of PK parameters between ubrogepant coadministration with erenumab as compared to erenumab alone is shown in Table 2.

TABLE 2
Comparison of PK parameters: Ubrogepant Coadministration
with Erenumab vs. Ubrogepant Alone

| | Geometric LSM | | Ratio of | 90% | 90% |
|---|---|---|---|---|---|
| PK | Ubrogepant | Ubrogepant | Geometric | lower | Upper |
| Parameter | Alone | and Erenumab | Means | CI | CI |
| Cₘₐₓ, | 430.9 | 447.6 | 1.04 | 0.93 | 1.16 |
| ng/mL | | | | | |
| AUC₀₋ₜ, | 1776.2 | 1874.8 | 1.06 | 0.96 | 1.16 |
| ng · h/mL | | | | | |
| AUC_{0-∞}, | 1812.4 | 1970.0 | 1.05 | 0.96 | 1.15 |
| ng · h/mL | | | | | |

AUC_{0-∞}, area under the plasma concentration-time curve from time 0 to infinity; AUC₀₋ₜ, area under the concentration-time curve from time 0 to t; Cₘₐₓ, peak plasma concentration; LSM, least squares mean; PK, pharmacokinetic.

As shown in Table 2, there was no significant change in ubrogepant Cₘₐₓ, AUC₀₋ₜ, or AUC_{0-∞} after coadministration with erenumab versus being administered alone.

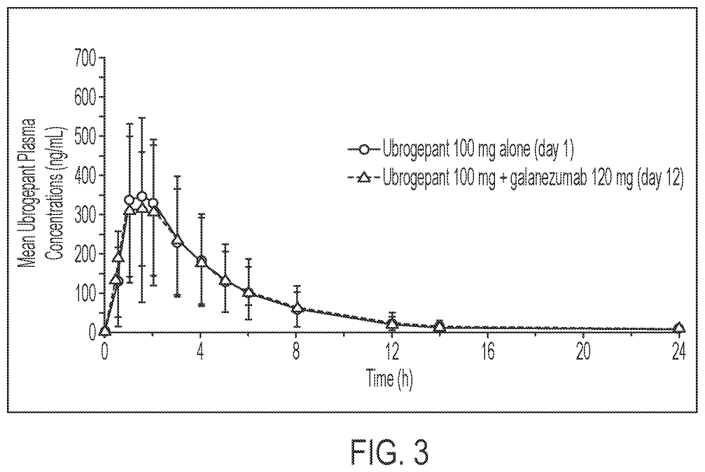

Mean ubrogepant plasma concentration-time profiles with and without coadministration of galcanezumab are presented in FIG. 3. Mean PK parameters are shown in Table 3.

TABLE 3
Ubrogepant Pharmacokinetics: Coadministration
with Galcanezumab

| PK Parameter, | Ubrogepant Alone | | Ubrogepant + Galcanezumab | |
|---|---|---|---|---|
| mean (SD) | (n = 20) | | (n = 20) | |
| C_{max,} ng/mL | 415.3 | (225.6) | 375.1 | (152.1) |
| AUC₀₋ₜ, ng · h/mL | 1700.3 | (913.4) | 1758.1 | (1033.4) |
| AUC_{0-∞}, ng · h/mL | 1732.2 | (928.1) | 1793.7 | (1057.0) |
| tₘₐₓ, h^{a} | 1.5 | (1.0-6.0) | 1.5 | (1.0-6.0) |
| t_{1/2,} h | 5.0 | (1.5) | 4.6 | (1.4) |
| V_{Z}/F, L | 568.7 | (497.9) | 449.3 | (213.8) |
| CL/F, L/h | 72.9 | (38.1) | 68.9 | (28.1) |

AUC_{0-∞}, area under the plasma concentration-time curve from time 0 to infinity; AUC₀₋ₜ, area under the concentration-time curve from time 0 to t; CL/F, apparent total body clearance of drug from plasma after extravascular administration; Cₘₐₓ, peak plasma concentration; PK, pharmacokinetic; t_{1/2}, elimination half-life; tₘₐₓ, time to peak plasma concentration; V_{Z}/F, apparent volume of distribution during the terminal phase after extravascular administration.

There was no significant change in ubrogepant Cₘₐₓ, AUC₀₋ₜ, or AUC₀₋₂₈ , after coadministration with galcanezumab versus being administered alone, as shown in Table 4.

TABLE 4
Comparison of PK Parameters: Ubrogepant Coadministration
with Galcanezumab vs. Ubrogepant alone

| | Geometric LSM | | Ratio of | 90% | 90% |
|---|---|---|---|---|---|
| PK | Ubrogepant | Ubrogepant and | Geometric | lower | Upper |
| Parameter | Alone | Galcanezumab | Means | CI | CI |
| Cₘₐₓ, | 358.7 | 357.0 | 1.00 | 0.82 | 1.20 |
| ng/mL | | | | | |
| AUC₀₋ₜ, | 1508.0 | 1583.9 | 1.05 | 0.90 | 1.23 |
| ng · h/mL | | | | | |
| AUC_{0-∞}, | 1541.3 | 1614.6 | 1.05 | 0.90 | 1.22 |
| ng · h/mL | | | | | |

AUC_{0-∞}, area under the plasma concentration-time curve from time 0 to infinity; AUC₀₋ₜ, area under the concentration-time curve from time 0 to t; Cₘₐₓ, peak plasma concentration; LSM, least squares mean; PK, pharmacokinetic.

Table 5 summarizes the PK parameters discussed above for ubrogepant administered alone or with erenumab or galcanezumab.

TABLE 5
PK Parameters for Ubrogepant Administered Alone or with Erenumab or Galcanezumab

| | Erenumab Arm | | | Galcanezumab Arm | | |
|---|---|---|---|---|---|---|
| PK | Ubrogepant | | | Ubrogepant | | |
| parameter, | Alone | Ubrogepant + | GMR | Alone | Ubrogepant + | GMR |
| GLSM | n = 20 | Erenumab | [90% CI] | n = 20 | Galcanezumab | [90% CI] |
| Cₘₐₓ, | 430.9 | 447.6 | 1.04 | 358.7 | 357.0 | 1.00 |
| ng/mL | | | [0.93, 1.16] | | | [0.82, 1.20] |
| AUC₀₋ₜ, | 1776.2 | 1874.8 | 1.06 | 1508.0 | 1583.9 | 1.05 |
| ng · h/mL | | | [0.96, 1.16] | | | [0.90, 1.23] |
| AUC_{0-∞}, | 1812.4 | 1910.0 | 1.05 | 1541.3 | 1614.6 | 1.05 |
| ng · h/mL | | | [0.96, 1.15] | | | [0.90, 1.22] |

As shown in Table 5, ubrogepant Cₘₐₓ, AUC₀₋ₜ, and AUC_{0-∞}, were minimally elevated after erenumab administration. With and without erenumab, median ubrogepant Tₘₐₓ was 1.5 hours; t_{1/2} was similar with (5.3 h) and without (4.6 h) erenumab.

As shown in Table 5, with galcanezumab coadministration, ubrogepant Cₘₐₓ was unchanged while AUC₀₋ₜ, and AUC_{0-∞}, were 5% higher. Ubrogepant Tₘₐₓ was 1.5 hours; t_{1/2} was similar with (5.0 h) and without (4.6h) galcanezumab.

A summary of treatment emergent adverse events (TEAEs) and treatment-related adverse events following all three study interventions of each arm is shown in Table 6.

TABLE 6
Overview of Adverse Events

| | | Ubrogepant Only | | Anti-CGRP | | Ubrogepant QD for | |
|---|---|---|---|---|---|---|---|
| | | 100 mg | | Monoclonal | | 4 Days Following | |
| | | Single Dose | | Antibody Only | | Monoclonal Antibody | |
| | | (n = 20) | | (n = 19)^{a} | | (n = 19) | |
| | Summary of AEs, | Timing of AE Collection | | | | | |
| | Participants (%) | Days 1-7 | | Days 8-11 | | Days 12-16+ | |
| Erenumab | TEAEs | 7 | (35.0) | 8 | (42.1) | 7 | (36.8) |
| Arm | Treatment Related AEs | 2 | (10.0) | 7 | (36.8) | 5 | (26.3) |
| | Headache | 1 | (5.0) | 6 | (31.6) | 1 | (5.3) |
| | Nausea | 0 | | 0 | | 2 | (10.5) |
| | Constipation | 0 | | 0 | | 1 | (5.3) |
| | Somnolence | 1 | (5.0) | 0 | | 0 | |
| | Flatulence | 0 | | 1 | (5.3) | 0 | |
| | Nasopharyngitis | 1 | (5.0) | 0 | | 0 | |
| | Abdominal Pain | 0 | | 0 | | 1 | (5.3) |
| | Upper Abdominal Pain | 0 | | 0 | | 1 | (5.3) |
| Galcanezumab | TEAEs | 2 | (10.0) | 3 | (15.0) | 4 | (21.1) |

| | | | | | | |
|---|---|---|---|---|---|---|
| Arm | Treatment-related AEs | 0 | 3 | (15.0) | 3 | (15.8) |

| | | | | |
|---|---|---|---|---|
| Headache | 0 | 3 | (15.0) | 0 |
| Hiccups | 0 | 1 | (5.0) | 0 |

| | | | | |
|---|---|---|---|---|
| Abdominal Discomfort | 0 | 0 | 1 | (5.3) |
| Upper Abdominal Pain | 0 | 0 | 1 | (5.3) |
| Dizziness | 0 | 0 | 1 | (5.3) |

^{a}20 participants in the galcanezumab arm
AE, adverse event; CGRP, calcitonin gene-related peptide; QD, once daily; TEAE, treatment-emergent adverse event.

No serious adverse events (AEs), AEs leading to discontinuation, or deaths were reported when ubrogepant was coadministered with erenumab or galcanezumab. In the erenumab arm, treatment-emergent AEs (TEAEs) were reported by 7/20 participants receiving ubrogepant alone, 8/19 participants with erenumab, and 7/19 with ubrogepant and erenumab; treatment-related AEs were reported by 2/20, 7/19, and 5/19, respectively. In the galcanezumab arm, TEAEs were reported by 2/20 participants receiving ubrogepant alone, 3/20 with galcanezumab, and 4/19 with ubrogepant and galcanezumab; treatment-related AEs were reported by 0/20, 3/20, and 3/19, respectively. No clinically relevant changes in laboratory parameters, vital signs, or ECG values were observed. The most common specific AE following any study intervention was headache in 6 participants following erenumab injection (days 8-11). One participant reported multiple GI treatment-related AEs during the erenumab study. These included flatulence (day 8), constipation (days 12-17), and abdominal pain (days 14-15).

In summary, ubrogepant PK was unchanged when co-administered with erenumab or galcanezumab, and no safety concerns were identified. Ubrogepant appears safe and well-tolerated in people receiving monoclonal antibody CGRP targeted therapies for preventive treatment of migraine.

### EMBODIMENTS

1. A method for treating, preventing, alleviating or reducing the frequency of occurrence of headache in a patient in need thereof, comprising administering to the patient a first antagonist of calcitonin gene-related peptide (CGRP-antagonist), wherein said patient is concurrently undergoing treatment with a second CGRP-antagonist.
2. A method for treating, preventing, alleviating or reducing the frequency of occurrence of headache in a patient in need thereof, comprising administering to the patient;
(a) a first antagonist of calcitonin gene-related peptide (CGRP-antagonist); and,
(b) second CGRP-antagonist.
3. A method for treating, preventing, alleviating or reducing the frequency of occurrence of migraine or a symptom of migraine in a patient in need thereof, comprising administering to the patient a first CGRP-antagonist, wherein said patient is concurrently undergoing treatment with a second CGRP-antagonist.
4. A method for treating, preventing, alleviating or reducing the frequency of occurrence of migraine or a symptom of migraine in a patient in need thereof, comprising administering to the patient;
(a) a first CGRP-antagonist; and,
(b) a second CGRP-antagonist.
5. The method according to claim 3 or 4 wherein the headache is a migraine headache, a cluster headache, a hemicrania continua headache, a chronic headache, a tension headache, a chronic tension headache, or any combination thereof
6. The method according to any of claims 1-4 wherein said first CGRP-antagonist is an antibody.
7. The method according to claim 6 wherein said antibody is selected from galcanezumab, fremanezumab, eptinezumab, and erenumab.
8. The method according to any of the above claims wherein said second CGRP-antagonist is selected from ubrogepant, atogepant and rimegepant, or a pharmaceutically acceptable salt, ester or prodrug thereof.
9. The method according to claim 6 wherein said antibody is galcanezumab.
10. The method according to claim 6 wherein said antibody is erenumab.
11. The method according to any of the above claims wherein said second CGRP-antagonist is selected from ubrogepant, atogepant, rimegepant or a pharmaceutically acceptable salt thereof.
12. The method according to any of claims 3-11 wherein said patient experiences reduced frequency of one or more symptoms or side effects of migraine selected from sinusitis, nausea, nasopharyngitis in comparison with a patient undergoing treatment with the first CGRP-antagonist alone.
13. The method according to any of claims 3-12 wherein the frequency of symptoms associated with one or more stages of migraine selected from prodrome, aura, headache or postdrome is reduced.
14. The method according to claim 13 wherein one or more frequency of symptoms associated with prodrome stage of migraine selected from photophobia, appetite changes, cognition and concentration difficulties, cold extremities, diarrhea or other bowel changes, excitement or irritability, fatigue, frequent urination, memory changes, weakness, yawning and stretching is reduced.
15. The method according to claim 13 wherein one or more frequency of symptoms associated with aura stage of migraine selected from seeing bright spots or flashes of light, vision loss, seeing dark spots, tingling sensations, speech problems, aphasia, and tinnitus is reduced.
16. The method according to claim 13 wherein one or more frequency of symptoms associated with attack stage of migraine selected from photophobia, gastric stasis, pulsating or throbbing pain on one or both sides of the head, extreme sensitivity to light, sounds, or smells, worsening pain during physical activity, nausea and vomiting, abdominal pain or heartburn, loss of appetite, lightheadedness, blurred vision, and fainting is reduced.
17. The method according to any of claims 3-13 wherein said migraine is migraine without aura stage.
18. The method according to any of the about claims wherein said migraine is menstrual migraine, familial hemiplegic migraine or chronic migraine.
19. The method according to any of claims 6-18 wherein said anti-CGRP antibody or fragment thereof is administered at a dosage that is about 20% or 30% or 40% or 50% or 60% or 70% or 80% lower than the recommended dosage for the anti-CGRP antibody monotherapy.
20. The method according to any of claims 10-19 wherein said anti-calcitonin gene-related peptide receptor antibody or antigen-binding fragment thereof is administered to a peripheral nerve, a cranial nerve, a spinal nerve or combinations thereof.
21. The method according to any of the above claims wherein said patient is administered one or more additional medications for the treatment of headache.
22. The method according to claim 21 wherein said additional medication is selected from opioid analgesics, Cox-2 inhibitors, barbiturates, sodium channel modulators, potassium channel modulators, calcium channel blockers, local anesthetics, monoamine oxidase inhibitors, leukotriene LTD4 receptor blocker, substance P antagonists, 5-HT3 antagonists and NMDA antagonists.
23. The method according to claim 21 wherein said one or more additional medications is selected from aspirin, ibuprofen, naproxen, acetaminophen, diclofenac, flurbiprofen, meclofenamate, isometheptene, indomethacin; codeine, morphine, hydrocodone, acetyldihydrocodeine, oxycodone, oxymorphone, papaverine, fentanyl, alfentanil, sufentanil, remifentanil, tramadol, prochlorperazine, celecoxib, rofecoxib, meloxicam, piroxicam, JTE-522, L-745,337, NS398, deracoxib, valdecoxib, lumiracoxib, etoricoxib, parecoxib, 4-(4-cyclohexyl-2-methyloxazol-5-yl)-2 fluorobenzenesulfonamide, (2-(3,5-difluorophenyl)-3-(4-(methylsulfonyl)phenyl)-2 cyclopenten-1-one, N-[2-(cyclohexyloxy)-4-nitrophenyl]methanesulfonamide, 2-(3,4 difluorophenyl)-4-(3-hydroxy-3-methylbutoxy)-5[4-(methylsulfonyl) phenyl]-3(2H) pyridazinone, 2-[(2,4-dichloro-6-methylphenyl) amino]-5-ethyl-benzeneacetic acid, (3Z) 3-[(4-chlorophenyl) [4-(methylsulfonyl)phenyl]methylene]dihydro-2(3H)-furanone, (S)-6,8-dichloro-2-(trifluoromethyl)-2H-1-benzopyran-3-carboxylic acid, amobarbital, butalbital, cyclobarbital, pentobarbital, allobarbital, methylphenobarbital, phenobarbital, secobarbital, vinylbital, verapamil, diltiazem, Nifedipine, lidocaine, tetracaine, prilocaine, bupivacaine, mepivacaine, etidocaine, procaine, benzocaine, phenelzine, isocarboxazid, dichloralphenazone, nimodipine, metoclopramide, capsaicin receptor agonists, captopril, tiospirone, a steroid, caffeine, metoclopramide, domperidone, scopolamine, dimenhydrinate, diphenhydramine, hydroxyzine, diazepam, lorazepam, chlorpromazine, methotrimeprazine, perphenazine, prochlorperazine, promethazine, trifluoperazine, triflupromazine, benzquinamide, bismuth sub salicylate, buclizine, cinnarizine, cyclizine, diphenidol, dolasetron, domperidone, dronabinol, droperidol, haloperidol, metoclopramide, nabilone, thiethylperazine, trimethobenzamide, and eziopitant, Meclizine, domperidone, ondansetron, tropisetron granisetron dolasetron, hydrodolasetron, palonosetron, alosetron, cilansetron, cisapride, renzapride metoclopramide, galanolactone, phencyclidine, ketamine, dextromethorphan, and isomers, pharmaceutically acceptable salts, esters, conjugates, or prodrugs thereof.
24. The method according to any of claims 6-23 wherein said anti-CGRP antibody is erenumab.
25. The method according to claim 24 wherein erenumab is administered subcutaneously at a dose of about 5 mg to about 500 mg every one, two, three, four, five, six, seven, eight, nine or ten weeks.
26. The method according to claim 24 wherein erenumab is administered subcutaneously at a dose of about 10 mg to about 200 mg every one, two, three, four, five, six, seven, eight, nine or ten weeks.
27. The method according to claim 24 wherein erenumab is administered subcutaneously at a dose of about 25 mg to about 150 mg every one, two, three, four, five, six, seven, eight, nine or ten weeks.
28. The method according to claim 24 wherein erenumab is administered subcutaneously at a dose of about 90 mg to about 120 mg every one, two, three, four, five, six, seven, eight, nine or ten weeks.
29. The method according to claim 24 wherein erenumab is administered subcutaneously at a dose of about 50 mg to about 60 mg every one, two, three, four, five, six, seven, eight, nine or ten weeks.
30. The method according to claim 24 wherein erenumab is administered subcutaneously at a dose of about 70 mg every one, two, three, four, five, six, seven, eight, nine or ten weeks.
31. The method according to claim 24 wherein erenumab is administered subcutaneously at a dose of about 140 mg every one, two, three, four, five, six, seven, eight, nine or ten weeks.
32. The method according to claim 24 wherein erenumab is administered subcutaneously at a monthly dose of about 140 mg.
33. The method according to claim 24 wherein erenumab is administered subcutaneously at a monthly dose of about 70 mg.
34. The method according to claim 24 wherein erenumab is administered subcutaneously at a dose of about 140 mg every two months.
35. The method according to claim 24 wherein erenumab is administered subcutaneously at a dose of about 70 mg every two months.
36. The method according to claim 24 wherein erenumab is administered subcutaneously at a dose of about 140 mg every three months.
37. The method according to claim 24 wherein erenumab is administered subcutaneously at a dose of about 70 mg every three months.
38. The method according to any of claims 6-23 wherein said anti-CGRP antibody is galcanezumab.
39. The method according to claim 38 wherein galcanezumab is administered subcutaneously at a dose of about 10 mg to about 500 mg every one, two, three, four, five, six, seven, eight, nine or ten weeks.
40. The method according to claim 38 wherein galcanezumab is administered subcutaneously at a dose of about 50 mg to about 300 mg every one, two, three, four, five, six, seven, eight, nine or ten weeks.
41. The method according to claim 38 wherein galcanezumab is administered subcutaneously at a dose of about 75 mg to about 250 mg every one, two, three, four, five, six, seven, eight, nine or ten weeks.
42. The method according to claim 38 wherein galcanezumab is administered subcutaneously at a dose of about 75 mg to about 100 mg every one, two, three, four, five, six, seven, eight, nine or ten weeks.
43. The method according to claim 38 wherein galcanezumab is administered subcutaneously at a dose of about 150 mg to about 220 mg every one, two, three, four, five, six, seven, eight, nine or ten weeks.
44. The method according to claim 38 wherein galcanezumab is administered subcutaneously at a dose of about 120 mg every one, two, three, four, five, six, seven, eight, nine or ten weeks.
45. The method according to claim 38 wherein galcanezumab is administered subcutaneously at a dose of about 240 mg every one, two, three, four, five, six, seven, eight, nine or ten weeks.
46. The method according to claim 38 wherein galcanezumab is administered subcutaneously at a monthly dose of about 240 mg.
47. The method according to claim 38 wherein galcanezumab is administered subcutaneously at a monthly dose of about 120 mg.
48. The method according to claim 38 wherein galcanezumab is administered subcutaneously at a dose of about 240 mg every two months.
49. The method according to claim 38 wherein galcanezumab is administered subcutaneously at a dose of about 120 mg every two months.
50. The method according to claim 38 wherein galcanezumab is administered subcutaneously at a dose of about 240 mg every three months.
51. The method according to claim 38 wherein galcanezumab is administered subcutaneously at a dose of about 120 mg every three months.
52. The method according to any of claims 6-23 wherein said anti-CGRP antibody is fremanezumab.
53. The method according to claim 52 wherein fremanezumab is administered subcutaneously at a dose of about 100 mg to about 1000 mg every one, two, three, four, five, six, seven, eight, nine or ten weeks.
54. The method according to claim 52 wherein fremanezumab is administered subcutaneously at a dose of about 150 mg to about 700 mg every one, two, three, four, five, six, seven, eight, nine or ten weeks.
55. The method according to claim 52 wherein fremanezumab is administered subcutaneously at a dose of about 150 mg to about 500 mg every one, two, three, four, five, six, seven, eight, nine or ten weeks.
56. The method according to claim 52 wherein fremanezumab is administered subcutaneously at a dose of about 150 mg to about 200 mg every one, two, three, four, five, six, seven, eight, nine or ten weeks.
57. The method according to claim 52 wherein fremanezumab is administered subcutaneously at a dose of about 150 mg to about 500 mg every one, two, three, four, five, six, seven, eight, nine or ten weeks.
58. The method according to claim 52 wherein fremanezumab is administered subcutaneously at a dose of about 225 mg every one, two, three, four, five, six, seven, eight, nine or ten weeks.
59. The method according to claim 52 wherein fremanezumab is administered subcutaneously at a dose of about 450 mg every one, two, three, four, five, six, seven, eight, nine or ten weeks.
60. The method according to claim 52 wherein fremanezumab is administered subcutaneously at a dose of about 675 mg every one, two, three, four, five, six, seven, eight, nine or ten weeks.
61. The method according to claim 52 wherein fremanezumab is administered subcutaneously at a monthly dose of about 225 mg.
62. The method according to claim 52 wherein fremanezumab is administered subcutaneously at a monthly dose of about 450 mg.
63. The method according to claim 52 wherein fremanezumab is administered subcutaneously at a monthly dose of about 675 mg.
64. The method according to claim 52 wherein fremanezumab is administered subcutaneously at a dose of about 225 mg every two months.
65. The method according to claim 52 wherein fremanezumab is administered subcutaneously at a dose of about 450 mg every two months.
66. The method according to claim 52 wherein fremanezumab is administered subcutaneously at a dose of about 225 mg every three months.
67. The method according to claim 52 wherein fremanezumab is administered subcutaneously at a dose of about 450 mg every three months.
68. The method according to claim 52 wherein fremanezumab is administered subcutaneously at a dose of about 675 mg every three months.
69. The method according to any of claims 6-23 wherein said anti-CGRP antibody is eptinezumab.
70. The method according to claim 69 wherein eptinezumab is administered subcutaneously at a dose of about 50 mg to about 1000 mg every one, two, three, four, five, six, seven, eight, nine or ten weeks.
71. The method according to claim 69 wherein eptinezumab is administered subcutaneously at a dose of about 100 mg to about 710 mg every one, two, three, four, five, six, seven, eight, nine or ten weeks.
72. The method according to claim 69 wherein eptinezumab is administered subcutaneously at a dose of about 200 mg to about 500 mg every one, two, three, four, five, six, seven, eight, nine or ten weeks.
73. The method according to claim 69 wherein eptinezumab is administered subcutaneously at a dose of about 250 mg to about 350 mg every one, two, three, four, five, six, seven, eight, nine or ten weeks.
74. The method according to claim 69 wherein eptinezumab is administered subcutaneously at a dose of about 300 mg every one, two, three, four, five, six, seven, eight, nine or ten weeks.
75. The method according to claim 69 wherein eptinezumab is administered subcutaneously at a monthly dose of about 100 mg.
76. The method according to claim 69 wherein eptinezumab is administered subcutaneously at a monthly dose of about 200 mg.
77. The method according to claim 69 wherein eptinezumab is administered subcutaneously at a monthly dose of about 300 mg.
78. The method according to claim 69 wherein eptinezumab is administered subcutaneously at a dose of about 100 mg every two months.
79. The method according to claim 69 wherein eptinezumab is administered subcutaneously at a dose of about 200 mg every two months.
80. The method according to claim 69 wherein eptinezumab is administered subcutaneously at a dose of about 300 mg every two months.
81. The method according to claim 69 wherein eptinezumab is administered subcutaneously at a dose of about 100 mg every three months.
82. The method according to claim 69 wherein eptinezumab is administered subcutaneously at a dose of about 200 mg every three months.
83. The method according to claim 69 wherein eptinezumab is administered subcutaneously at a dose of about 300 mg every three months.
84. The method according to any of claims wherein said migraine is chronic migraine.
85. The method according to any of claims wherein said migraine is episodic migraine.
86. The method according to any of the above claims wherein said CGRP-antagonist a) bind to CGRP or CGRP-R and the binding results in a reduction or inhibition of CGRP activity; (b) block CGRP from binding to its receptor(s); (c) block or decrease CGRP receptor activation; (d) inhibit CGRP biological activity or downstream pathways mediated by CGRP signaling function; (e) increase clearance of CGRP; or (f) inhibit or reduce CGRP synthesis, production or release.
87. An improved method for treating, preventing, alleviating or reducing the frequency of occurrence of headache in a patient in need thereof, wherein the improvement comprises administering to the patient a long acting antagonist of calcitonin gene-related peptide (long acting CGRP-antagonist), wherein said patient is concurrently undergoing treatment with a short acting antagonist of calcitonin gene-related peptide (short acting CGRP-antagonist).
88. An improved method for treating, preventing, alleviating or reducing the frequency of occurrence of headache in a patient in need thereof, the improvement comprises administering to the patient;
(a) long acting CGRP-antagonist; and,
(b) short acting CGRP-antagonist.
89. The method according to claim 87 or 88 wherein the headache is a migraine headache, a cluster headache, a hemicrania continua headache, a chronic headache, a tension headache, a chronic tension headache, a post traumatic headache, a post-ictal headache, or any combination thereof.
90. The method according to claim 87-89 wherein said long acting CGRP-antagonist is an antibody is selected from galcanezumab, fremanezumab, eptinezumab, and erenumab.
91. The method according to any of claims 87-90 wherein said short acting CGRP-antagonist is selected from ubrogepant, atogepant, rimegepant or a pharmaceutically acceptable salt thereof.
92. The method according to any of claims 1-86 wherein said first CGRP-antagonist is administered parenterally, preferably intravenously.
93. The method according to any of claims 1-92 wherein said second CGRP-antagonist is ubrogepant or a pharmaceutically acceptable salt thereof, and ubrogepant is administered at a dose of about 5 to about 500 mg per day.
94. The method according to claim 93 wherein said dose is about 5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190 or 200 mg per day.
95. The method according to any of claims 1-92 wherein said second CGRP-antagonist is atogepant or a pharmaceutically acceptable salt thereof, and atogepant is administered at a dose of about 5 to about 500 mg per day.
96. The method according to claim 95 wherein said dose is about 5, 10, 15, 20, 25, 30, 35, 40, 45, 50, 55, 60, 65, 70, 75, 80, 85, 90, 95, 100, 110, 120, 125, 130, 140, 150, 160, 170, 175, 180, 190 or 200 mg per day.
97. The method according to any of the above claims wherein one or more symptoms of migraine is reduced.
98. The method according to claim 97 wherein said symptom is associated with prodrome stage of migraine wherein said symptom is selected from photophobia, appetite changes, cognition and concentration difficulties, cold extremities, diarrhea or other bowel changes, excitement or irritability, fatigue, frequent urination, memory changes, weakness, yawning and stretching.
99. The method according to claim 98 wherein said symptom is associated with aura stage of migraine wherein said symptom is selected from seeing bright spots or flashes of light, vision loss, seeing dark spots, tingling sensations, speech problems, aphasia, and tinnitus.
100. The method according to claim 97 wherein said symptom is associated with attack stage of migraine wherein said symptom is selected from photophobia, gastric stasis, pulsating or throbbing pain on one or both sides of the head, extreme sensitivity to light, sounds, or smells, worsening pain during physical activity, nausea and vomiting, abdominal pain or heartburn, loss of appetite, lightheadedness, blurred vision, and fainting.

1. A method for treating, preventing, alleviating, or reducing the frequency of occurrence of headache in a patient in need thereof, comprising administering to the patient:
(a) a first calcitonin gene related peptide antagonist (CGRP antagonist) and
(b) a second CGRP-antagonist.

2. The method of claim 1, wherein the headache is migraine headache.

3. The method of claim 1, wherein the first CGRP antagonist is an antibody, and the second CGRP antagonist has a plasma half-life in humans of at most about 60 hours.

4. The method of claim 3, wherein the second CGRP antagonist has a plasma half-life in humans of at most about 12 hours.

5. The method of claim 3, wherein the first CGRP antagonist is an antibody that targets the CGRP receptor.

6. The method of claim 3, wherein the first CGRP antagonist is an antibody that targets the CGRP ligand.

7. The method of claim 1, wherein the first CGRP antagonist is an antibody selected from the group consisting of galcanezumab, fremanzeumab, eptinezumab, and erenumab.

8. The method of claim 1, wherein the second CGRP-antagonist is selected from the group consisting of ubrogepant, atogepant, and rimegepant.

9. The method of claim 1, wherein the first CGRP antagonist is an antibody selected from the group consisting of galcanezumab, fremanzumab, eptinezumab, and erenumab; and the second CGRP antagonist is selected from the group consisting of ubrogepant, atogepant, and rimegepant.

10. The method of claim 9, wherein the first CGRP antagonist is galcanezumab.

11. The method of claim 9, wherein the first CGRP antagonist is erenumab.

12. The method of claim 9, wherein the second CGRP antagonist is ubrogepant.

13. A method of treating, preventing, alleviating, or reducing the frequency of occurrence of headache in a patient in need thereof, the method comprising administering to the patient (a) erenumab and (b) ubrogepant.

14. The method according to claim 13, wherein erenumab is administered subcutaneously at a dose of about 140 mg.

15. The method according to claim 13, where ubrogepant is administered at a dose of about 5 to about 500 mg.

16. The method according to claim 15, wherein ubrogepant is administered at a dose of about 50 mg.

17. The method according to claim 15, wherein ubrogepant is administered at a dose of about 100 mg.

18. A method of treating, preventing, alleviating, or reducing the frequency of occurrence of headache in a patient in need thereof, the method comprising administering to the patient (a) galcanezumab and (b) ubrogepant.

19. The method according to claim 18, wherein galcanezumab is administered subcutaneously at a dose of about 240 mg.

20. The method according to claim 18, wherein ubrogepant is administered at a dose of about 5 to about 500 mg.

21. The method according to claim 20, wherein ubrogepant is administered at a dose of about 50 mg.

22. The method according to claim 20, wherein ubrogepant is administered at a dose of about 100 mg.
